SEQUENCE LISTING (110 sequences)

SEQ ID NO: 1 - protein, 12 aa, artificial sequence
  peptide retrieved from phage biopanning
AMAGTTSDPSTV

SEQ ID NO: 2 - protein, 12 aa, artificial sequence
  peptide retrieved from phage biopanning
AASPTQSMSQAP

SEQ ID NO: 3 - protein, 12 aa, artificial sequence
  peptide retrieved from phage biopanning
HTHTNNDSPNQA

SEQ ID NO: 4 - protein, 12 aa, artificial sequence
  peptide retrieved from phage biopanning
DTQGFHSRSSSA

SEQ ID NO: 5 - protein, 12 aa, artificial sequence
  peptide retrieved from phage biopanning
TSSSALQPAHAW

SEQ ID NO: 6 - protein, 12 aa, artificial sequence
  peptide retrieved from phage biopanning
SESSPISLDYRA

SEQ ID NO: 7 - protein, 12 aa, artificial sequence
  peptide retrieved from phage biopanning
STHNYQIPRPPT

SEQ ID NO: 8 - protein, 12 aa, artificial sequence
  peptide retrieved from phage biopanning
HPFSNEPLQLSS

SEQ ID NO: 9 - protein, 12 aa, artificial sequence
  peptide retrieved from phage biopanning
GTLANQQIFLSS

SEQ ID NO: 10 - protein, 12 aa, artificial sequence
  peptide retrieved from phage biopanning
HGNPLPMTPFPG

SEQ ID NO: 11 - protein, 12 aa, artificial sequence
  peptide retrieved from phage biopanning
RLELAIPLQGSG

SEQ ID NO: 12 - protein, 9 aa, artificial sequence
  peptide retrieved from phage biopanning
CHASNRLSC

SEQ ID NO: 13 - protein, 12 aa, artificial sequence
  peptide retrieved from phage biopanning
SMDRSDMTMRLP

SEQ ID NO: 14 - protein, 12 aa, artificial sequence
  peptide retrieved from phage biopanning
GTFTPRPTPIYP

SEQ ID NO: 15 - protein, 12 aa, artificial sequence
  peptide retrieved from phage biopanning
QMSENLTSQIES

SEQ ID NO: 16 - protein, 12 aa, artificial sequence
  peptide retrieved from phage biopanning
DMLARLRATAGP

SEQ ID NO: 17 - protein, 12 aa, artificial sequence
  peptide retrieved from phage biopanning
SQTWLLMSPVAT

SEQ ID NO: 18 - protein, 12 aa, artificial sequence
  peptide retrieved from phage biopanning
ASPDQQVGPLYV

SEQ ID NO: 19 - protein, 12 aa, artificial sequence
  peptide retrieved from phage biopanning
LTWSPLQTVARF

SEQ ID NO: 20 - protein, 12 aa, artificial sequence
  peptide retrieved from phage biopanning
QISAHQMPSRPI

SEQ ID NO: 21 - protein, 12 aa, artificial sequence
  peptide retrieved from phage biopanning
SMKYNLIVDSPY

SEQ ID NO: 22 - protein, 12 aa, artificial sequence
  peptide retrieved from phage biopanning
QMPIRNQLAWPM

SEQ ID NO: 23 - protein, 12 aa, artificial sequence
  peptide retrieved from phage biopanning
TQNLEIREPLTP

SEQ ID NO: 24 - protein, 12 aa, artificial sequence
  peptide retrieved from phage biopanning
YPMSPSPYPYQL

SEQ ID NO: 25 - protein, 12 aa, artificial sequence
  peptide retrieved from phage biopanning
SFMIQPTPLPPS

SEQ ID NO: 26 - protein, 12 aa, artificial sequence
  peptide retrieved from phage biopanning
GLAPHIHSLNEA

SEQ ID NO: 27 - protein, 12 aa, artificial sequence
  peptide retrieved from phage biopanning
MQFPVTPYLNAS

SEQ ID NO: 28 - protein, 12 aa, artificial sequence
  peptide retrieved from phage biopanning
SPGDSLKKLAAS

SEQ ID NO: 29 - protein, 12 aa, artificial sequence
  peptide retrieved from phage biopanning
GYHMQTLPGPVA

SEQ ID NO: 30 - protein, 12 aa, artificial sequence
  peptide retrieved from phage biopanning
SLTPLTTSHLRS

SEQ ID NO: 31 - protein, 12 aa, artificial sequence
  peptide retrieved from phage biopanning
TLTNGPLRPFTG

SEQ ID NO: 32 - protein, 12 aa, artificial sequence
  peptide retrieved from phage biopanning
LNTPKPFTLGQN

SEQ ID NO: 33 - protein, 9 aa, artificial sequence
  peptide retrieved from phage biopanning
CDLQNYKAC

SEQ ID NO: 34 - protein, 9 aa, artificial sequence
  peptide retrieved from phage biopanning
CRHPHTRLC

SEQ ID NO: 35 - protein, 9 aa, artificial sequence
  peptide retrieved from phage biopanning
CANLKPKAC

SEQ ID NO: 36 - protein, 9 aa, artificial sequence
  peptide retrieved from phage biopanning
CYINPPKVC

SEQ ID NO: 37 - protein, 9 aa, artificial sequence
  peptide retrieved from phage biopanning
CNNKVPVLC

SEQ ID NO: 38 - protein, 9 aa, artificial sequence
  peptide retrieved from phage biopanning
CHASKTPLC

SEQ ID NO: 39 - protein, 9 aa, artificial sequence
  peptide retrieved from phage biopanning
CASQLYPAC

SEQ ID NO: 40 - protein, 9 aa, artificial sequence
  peptide retrieved from phage biopanning
CNMTQYPAC

SEQ ID NO: 41 - protein, 9 aa, artificial sequence
  peptide retrieved from phage biopanning
CFAPSGPAC

SEQ ID NO: 42 - protein, 9 aa, artificial sequence
  peptide retrieved from phage biopanning
CPVWIQAPC

SEQ ID NO: 43 - protein, 9 aa, artificial sequence
  peptide retrieved from phage biopanning
CQVAVNPLC

SEQ ID NO: 44 - protein, 9 aa, artificial sequence
  peptide retrieved from phage biopanning
CQPEAMPAC

SEQ ID NO: 45 - protein, 9 aa, artificial sequence
  peptide retrieved from phage biopanning
CHPTMPLAC

SEQ ID NO: 46 - protein, 9 aa, artificial sequence
  peptide retrieved from phage biopanning
CPPFAAPIC

SEQ ID NO: 47 - protein, 9 aa, artificial sequence
  peptide retrieved from phage biopanning
CNKHQPMHC

SEQ ID NO: 48 - protein, 9 aa, artificial sequence
  peptide retrieved from phage biopanning
CFPMRSNQC

SEQ ID NO: 49 - protein, 9 aa, artificial sequence
  peptide retrieved from phage biopanning
CQSMPHNRC

SEQ ID NO: 50 - protein, 9 aa, artificial sequence
  peptide retrieved from phage biopanning
CNNPMHQNC

SEQ ID NO: 51 - protein, 9 aa, artificial sequence
  peptide retrieved from phage biopanning
CHMAPRWQC

SEQ ID NO: 52 - protein, 9 aa, artificial sequence
  peptide retrieved from phage biopanning
HVHIHSRPM

SEQ ID NO: 53 - protein, 9 aa, artificial sequence
  peptide retrieved from phage biopanning
LPNMHPLPL

SEQ ID NO: 54 - protein, 9 aa, artificial sequence
  peptide retrieved from phage biopanning
LPLRLPPMP

SEQ ID NO: 55 - protein, 9 aa, artificial sequence
  peptide retrieved from phage biopanning
HSMIGTPTT

SEQ ID NO: 56 - protein, 9 aa, artificial sequence
  peptide retrieved from phage biopanning
SVSVGMKPS

SEQ ID NO: 57 - protein, 9 aa, artificial sequence
  peptide retrieved from phage biopanning
LDASFMQDW

SEQ ID NO: 58 - protein, 9 aa, artificial sequence
  peptide retrieved from phage biopanning
TPPSYQMAM

SEQ ID NO: 59 - protein, 9 aa, artificial sequence
  peptide retrieved from phage biopanning
YPQLVSMST

SEQ ID NO: 60 - protein, 9 aa, artificial sequence
  peptide retrieved from phage biopanning
GYSTINMYS

SEQ ID NO: 61 - protein, 9 aa, artificial sequence
  peptide retrieved from phage biopanning
DRMLLPFNL

SEQ ID NO: 62 - protein, 9 aa, artificial sequence
  peptide retrieved from phage biopanning
IPMTPSYDS

SEQ ID NO: 63 - protein, 9 aa, artificial sequence
  peptide retrieved from phage biopanning
MYSPRPPAL

SEQ ID NO: 64 - protein, 9 aa, artificial sequence
  peptide retrieved from phage biopanning
QPTTDLMAH

SEQ ID NO: 65 - protein, 9 aa, artificial sequence
  peptide retrieved from phage biopanning
ATHVQMAWA

SEQ ID NO: 66 - protein, 9 aa, artificial sequence
  peptide retrieved from phage biopanning
SMHATLTPM

SEQ ID NO: 67 - protein, 9 aa, artificial sequence
  peptide retrieved from phage biopanning
SGPAHGMFA

SEQ ID NO: 68 - protein, 9 aa, artificial sequence
  peptide retrieved from phage biopanning
IANRPYSAQ

SEQ ID NO: 69 - protein, 7 aa, artificial sequence
  peptide retrieved from phage biopanning
VMTQPTR

SEQ ID NO: 70 - protein, 7 aa, artificial sequence
  peptide retrieved from phage biopanning
HMRPLSI

SEQ ID NO: 71 - protein, 12 aa, artificial sequence
  peptide retrieved from phage biopanning
LTRSPLHVDQRR

SEQ ID NO: 72 - protein, 12 aa, artificial sequence
  peptide retrieved from phage biopanning
VISNHAESSRRL

SEQ ID NO: 73 - protein, 7 aa, artificial sequence
  peptide retrieved from phage biopanning
HTHIPNQ

SEQ ID NO: 74 - protein, 7 aa, artificial sequence
  peptide retrieved from phage biopanning
LAPVSPP

SEQ ID NO: 75 - protein, 9 aa, artificial sequence
  peptide retrieved from phage biopanning
CMTAGKNTC

SEQ ID NO: 76 - protein, 9 aa, artificial sequence
  peptide retrieved from phage biopanning
CQTLWRNSC

SEQ ID NO: 77 - protein, 9 aa, artificial sequence
  peptide retrieved from phage biopanning
CTSVHTNTC

SEQ ID NO: 78 - protein, 9 aa, artificial sequence
  peptide retrieved from phage biopanning
CPSLAMNSC

SEQ ID NO: 79 - protein, 9 aa, artificial sequence
  peptide retrieved from phage biopanning
CSNNTVHAC

SEQ ID NO: 80 - protein, 9 aa, artificial sequence
  peptide retrieved from phage biopanning
CLPAQGHVC

SEQ ID NO: 81 - protein, 9 aa, artificial sequence
  peptide retrieved from phage biopanning
CLPAQVHVC

SEQ ID NO: 82 - protein, 9 aa, artificial sequence
  peptide retrieved from phage biopanning
CPPKNVRLC

SEQ ID NO: 83 - protein, 9 aa, artificial sequence
  peptide retrieved from phage biopanning
CPHINAHAC

SEQ ID NO: 84 - protein, 9 aa, artificial sequence
  peptide retrieved from phage biopanning
CIVNLARAC

SEQ ID NO: 85 - protein, 12 aa, artificial sequence
  peptide retrieved from phage biopanning
TMGFTAPRFPHY

SEQ ID NO: 86 - protein, 12 aa, artificial sequence
  peptide retrieved from phage biopanning
ATQSYVRHPSLG

SEQ ID NO: 87 - protein, 12 aa, artificial sequence
  peptide retrieved from phage biopanning
TSTTQGALAYLF

SEQ ID NO: 88 - protein, 12 aa, artificial sequence
  peptide retrieved from phage biopanning
DPPWSAIVRHRD

SEQ ID NO: 89 - protein, 12 aa, artificial sequence
  peptide retrieved from phage biopanning
FDNKPFLRVASE

SEQ ID NO: 90 - protein, 12 aa, artificial sequence
  peptide retrieved from phage biopanning
HQSHTQQNKRHL

SEQ ID NO: 91 - protein, 12 aa, artificial sequence
  peptide retrieved from phage biopanning
TSTTQGALAYLF

SEQ ID NO: 92 - protein, 12 aa, artificial sequence
  peptide retrieved from phage biopanning
KTPIHTSAWEFQ

SEQ ID NO: 93 - protein, 12 aa, artificial sequence
  peptide retrieved from phage biopanning
DLFHLKPVSNEK

SEQ ID NO: 94 - protein, 12 aa, artificial sequence
  peptide retrieved from phage biopanning
KPFWTSSPDVMT

SEQ ID NO: 95 - protein, 12 aa, artificial sequence
  peptide retrieved from phage biopanning
PWAATSKPPYSS

SEQ ID NO: 96 - protein, 9 aa, artificial sequence
  peptide retrieved from phage biopanning
CQNPMQTFC

SEQ ID NO: 97 - protein, 9 aa, artificial sequence
  peptide retrieved from phage biopanning
CNQLSTRPC

SEQ ID NO: 98 - protein, 9 aa, artificial sequence
  peptide retrieved from phage biopanning
CLQNRQSQC

SEQ ID NO: 99 - protein, 9 aa, artificial sequence
  peptide retrieved from phage biopanning
CQLQRQWNC

SEQ ID NO: 100 - protein, 9 aa, artificial sequence
  peptide retrieved from phage biopanning
CQVNSAHQC

SEQ ID NO: 101 - protein, 9 aa, artificial sequence
  peptide retrieved from phage biopanning
CFPMRSNQC

SEQ ID NO: 102 - protein, 9 aa, artificial sequence
  peptide retrieved from phage biopanning
CPPQPNRQC

SEQ ID NO: 103 - protein, 9 aa, artificial sequence
  peptide retrieved from phage biopanning
CQMPMQHNC

SEQ ID NO: 104 - protein, 9 aa, artificial sequence
  peptide retrieved from phage biopanning
CANVAQRNC

SEQ ID NO: 105 - protein, 8 aa, artificial sequence
  peptide retrieved from phage biopanning
NNPMHQNC

SEQ ID NO: 106 - protein, 12 aa, artificial sequence
  peptide retrieved from phage biopanning
SGPAHGMFARPL

SEQ ID NO: 107 - protein, 9 aa, artificial sequence
  peptide retrieved from phage biopanning
CTYSRLHLC

SEQ ID NO: 108 - protein, 9 aa, artificial sequence
  peptide retrieved from phage biopanning
CRPYNIHQC

SEQ ID NO: 109 - protein, 9 aa, artificial sequence
  peptide retrieved from phage biopanning
CPFKTAFPC

SEQ ID NO: 110 - protein, 7 aa, artificial sequence
  peptide retrieved from phage biopanning
XXPMHXX

(19) United States
(12) Patent Application Publication
(10) Pub. No.: US 2011/0097556 A1
(43) Pub. Date: Apr. 28, 2011

(54) BIOLOGICAL CONTROL OF NANOPARTICLE NUCLEATION, SHAPE AND CRYSTAL PHASE

(75) Inventors: Angela M. Belcher, Lexington, MA (US); Christine E. Flynn, Austin, TX (US)

(73) Assignee: Board of Regents, The University of Texas System

(21) Appl. No.: 11/839,923

(22) Filed: Aug. 16, 2007

Related U.S. Application Data
(63) Continuation of application No. 10/158,596, filed on May 30, 2002, now abandoned.
Application No. 10/158,596 is a continuation-in-part of application No. 10/155,883, filed on May 24, 2002, now abandoned.
(60) Provisional application No. 60/325,664, filed on Sep. 28, 2001.
(60) Provisional application No. 60/296,013, filed on Jun. 5, 2001.

(51) Int. Cl. B32B 3/10; C40B 40/10; C40B 40/02; C12N 7/00

(52) U.S. Cl. 428/195.1; 506/18; 506/14; 435/235.1

(57) ABSTRACT

The present invention includes compositions and methods for selective binding of amino acid oligomers to semiconductor materials. One form of the present invention is a method for controlling the particle size of the semiconductor materials by interacting an amino acid oligomer that specifically binds the material with solutions that can result in the formation of the material. The same method can be used to control the aspect ratio of the nanocrystal particles of the semiconductor material. Another form of the present invention is a method to create nanowires from the semiconductor material.

The research carried out in the subject application was supported in part by grants from the Army Research Office (DADD19-99-0155).

### FIELD OF THE INVENTION

The present invention is directed to the selective recognition of inorganic materials in general and specifically toward surface recognition of semiconductor materials using peptides. A nucleotide and/or amino acid sequence listing is incorporated by reference of the material on computer readable form.

### BACKGROUND OF THE INVENTION

This application is a continuation of U.S. patent application Ser. No. 10/158,596, which claims priority from Provisional Patent Application Ser. No. 60/325,664, filed on Sep. 28, 2001. U.S. application Ser. No. 10/158,596 also is a continuation-in-part and claims priority from U.S. patent application Ser. No. 10/155,883 filed May 24, 2002, which claims priority to U.S. Provisional Patent Application 60/296,013 filed Jun. 5, 2001.

In biological systems, organic molecules exhibit a remarkable level of control over the nucleation and mineral phase of inorganic materials such as calcium carbonate and silica, and over the assembly of crystallites and other nanoscale building blocks into complex structures required for biological function. This control could, in theory, be applied to materials with interesting electrical or optical properties.

Materials produced by biological processes are typically soft, and consist of a surprisingly simple collection of molecular building blocks (i.e., lipids, peptides, and nucleic acids) arranged in astoundingly complex architectures. Unlike the semiconductor industry, which relies on a serial lithographic processing approach for constructing the smallest features on an integrated circuit, living organisms execute their architectural “blueprints” using mostly non-covalent forces acting simultaneously upon many molecular components. Furthermore, these structures can often elegantly rearrange between two or more usable forms without changing any of the molecular constituents.

The use of “biological” materials to process the next generation of microelectronic devices provides a possible solution to resolving the limitations of traditional processing methods. The critical factors in this approach are identifying the appropriate compatibilities and combinations of biological-inorganic materials, and the synthesis of the appropriate building blocks.

### SUMMARY OF THE INVENTION

The present invention is based on the selection, production, isolation and characterization of organic polymers, e.g., peptides, with enhanced selectivity for binding to metal, semiconductor and metal oxide surfaces. The present invention uses combinatorial libraries, e.g., a phage display library, to cause directed molecular recognition of a target taking advantage of iterative rounds of peptide evolution. Peptides can be created and derived that bind to a wide range of semiconductor surfaces with high specificity. Furthermore, the invention allows for the selective isolation of organic recognition molecules that specifically recognize a specific crystallographic orientation, whether or not a composition of the structurally similar materials is used. Semiconductor materials that were tested and shown to successfully bind peptides include gallium arsenide, indium phosphate, gallium nitrate, zinc sulfide, aluminum arsenide, aluminum gallium arsenide, cadmium sulfide, cadmium selenide, zinc selenide, lead sulfide, boron nitride and silicon.

Semiconductor nanocrystals exhibit size and shape-dependent optical and electrical properties. These diverse properties result in their potential applications in a variety of devices such as light emitting diodes (LED), single electron transistors, photovoltaics, optical and magnetic memories, and diagnostic markers and sensors. Control of particle size, shape and phase is also critical in protective coatings such as car paint and in pigments such as house paints. The semiconductor materials may be engineered to be of certain shapes and sizes, wherein the optical and electrical properties of these semiconductor materials may best be exploited for use in numerous devices.

More particularly, the present invention may be described as a method for directed semiconductor formation including the steps of contacting a polymeric organic material that binds a predetermined face specificity semiconductor material with a first ion to create a semiconductor material precursor and adding a second ion to the semiconductor material precursor, wherein the polymeric organic material directs formation of the predetermined face specific semiconductor material. The polymeric organic material may include an amino acid oligomer or peptide, which may be on the surface of a bacteriophage as part of, e.g., a chimeric coat protein. The polymeric organic material may even be a nucleic acid oligomer and may be selected from a combinatorial library. The polymeric organic material may be an amino acid polymer of between about 7 and 20 amino acids. The present invention also encompasses a semiconductor material made using the method of the present invention.

Uses for the controlled crystals directed and grown using the materials and methods of the present invention include materials with novel optical, electronic and magnetic properties. As will be known to those of skill in the art, the detailed optical, electronic and magnetic properties may be directed by the formation of semiconductor crystal by, e.g., patterning the devices, which using the present invention may include layering or laying down patterns to create crystal formation in patterns, layers or even both.

Another use of the patterns and/or layers formed using the present invention is the formation of semiconductor devices for high density magnetic storage. Another design may be for the formation of transistors for use in, e.g., quantum computing. Yet another use for the patterns, designs and novel materials made with the present invention include imaging and imaging contrast agent for medical applications.

One such use for the directed formation of semiconductors and semiconductor crystals and designs include information storage based on quantum dot patterns, e.g., identification of friend or foe in military or even personnel situations. The quantum dots could be used to identify individual soldiers or personnel using identification in fabric, in armor or on the person. Alternatively, the dots may be used in coding the fabric of money. Yet another use for the present invention is to create bi and multi-functional peptides for drug delivery in trapping the drug to be delivered using the peptides of the present invention. Yet another use is for in vivo and vitro diagnostics based on gene or protein expression by drug trapping using the peptides to deliver a drug.

### BRIEF DESCRIPTION OF THE FIGURES

For a more complete understanding of the features and advantages of the present invention, reference is now made to the detailed description of the invention along with the accompanying figures in which corresponding numerals in the different figures refer to corresponding parts and in which:

FIG. 1 depicts selected random amino acid sequences in accordance with the present invention;

FIG. 2 depicts XPS spectra of structures in accordance with the present invention;

FIG. 3 depicts phage recognition of heterostructures in accordance with the present invention;

FIGS. 4-8 depict specific amino acid sequences in accordance with the present invention;

FIG. 9 depicts the peptide insert structure of the phage libraries in accordance with the present invention;

FIG. 10 depicts the various amino acid substitutions in the third and fourth rounds of selection in accordance with the present invention;

FIG. 11 depicts the amino acid substitutions after the fifth round of selection in accordance with the present invention; and

FIG. 12 depicts the nanowire made from the ZnS nanoparticles in accordance with the present invention.

### DETAILED DESCRIPTION OF THE INVENTION

Although making and using various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention, and do not delimit the scope of the invention.

The inventors have previously shown that peptides can bind to semiconductor material. This technique has been further developed into a means of nucleating nanoparticles and directing their self-assembly. The main features of the peptides are their ability to recognize and bind technologically important materials with face specificity, to nucleate size-constrained crystalline semiconductor materials, and to control the crystallographic phase of nucleated nanoparticles. The peptides can also control the aspect ratio of the materials and therefore, the optical properties.

Briefly, the facility with which biological systems assemble immensely complicated structure on an exceedingly minute scale has motivated a great deal of interest in the desire to identify non-biological systems that can behave in a similar fashion. Of particular value would be methods that could be applied to materials with interesting electronic or optical properties, but natural evolution has not selected for interactions between biomolecules and such materials.

The present invention is based on recognition that biological systems efficiently and accurately assemble nanoscale building blocks into complex and functionally sophisticated structures with high perfection, controlled size and compositional uniformity.

One method of providing a random organic polymer pool is using a Phage-display library, a combinatorial library of random peptides containing between 7 and 12 amino acids fused to the pIII coat protein of M13 coliphage, providing different peptides that are reactive with crystalline semiconductor structures. Five copies of the pIII coat protein are located on one end of the phage particle, accounting for 10-16 nm of the particle. The phage-display approach provides a physical linkage between the peptide substrate interaction and the DNA that encodes that interaction. The examples described here used as examples, five different single-crystal semiconductors: GaAs (100), GaAs (111)A, GaAs(111)B, InP(100) and Si(100) These substrates allowed for systematic evaluation of the peptide substrate interactions and confirmation of the general utility of the methodology of the present invention for different crystalline structures.

Using a Phage-display library, Protein sequences that successfully bound to the specific crystal were eluted from the surface, amplified by, e.g., a million-fold, and reacted against the substrate under more stringent conditions. This procedure was repeated between three and seven times to select the phage in the library with the most specific binding peptides. After, e.g., the third, fourth and fifth rounds of phage selection, crystal-specific phage were isolated and their DNA sequenced, identifying the peptide binding that is selective for the crystal composition (for example, binding to GaAs but not to Si) and crystalline face (for example, binding to (100) GaAs, but not to (111)B GaAs).

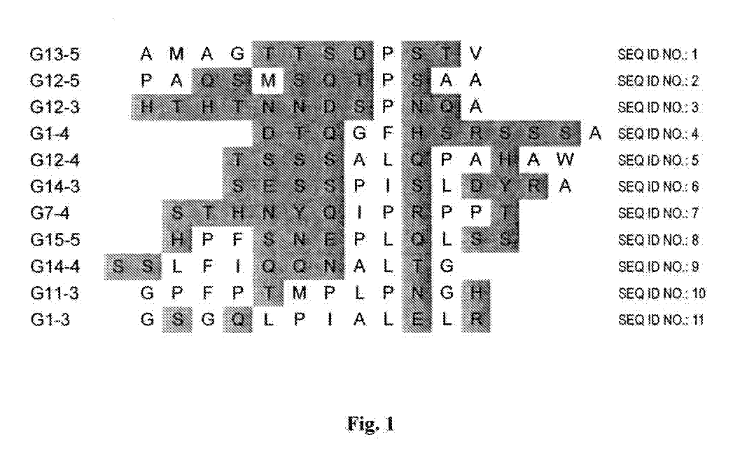

Twenty clones selected from GaAs(100) were analyzed to determine epitope binding domains by amino-acid functionality analysis to the GaAs surface. The partial peptide sequences of the modified pIII or pVIII protein are shown in FIG. 1, revealing similar binding domains among peptides exposed to GaAs. With increasing number of exposures to a GaAs surface, the number of uncharged polar and Lewis-base functional groups increased. Phage clones from third, fourth and fifth round sequencing contained on average 30%, 40% and 44% polar functional groups, respectively, while the fraction of Lewis-base functional groups increased at the same time from 41% to 48% to 55%. The observed increase in Lewis bases, which should constitute only 34% of the functional groups in random 12-mer peptides from our library, suggests that interactions between Lewis bases on the peptides and Lewis-acid sites on the GaAs surface may mediate the selective binding exhibited by these peptides.

The expected structure of the modified 12-mers selected from the library may be an extended conformation, which seems likely for small peptides, making the peptide much longer than the unit cell (5.65 A°) of GaAs. Therefore, only small binding domains would be necessary for the peptide to recognize a GaAs crystal. These short peptide domains, highlighted in FIG. 1, contain serine- and threonine-rich regions in addition to the presence of amine Lewis bases, such as asparagine and glutamine. To determine the exact binding sequence, the surfaces have been screened with shorter libraries, including 7-mer and disulphide constrained 7-mer libraries. Using these shorter libraries that reduce the size and flexibility of the binding domain, fewer peptide-surface interactions are allowed, yielding the expected increase in the strength of interactions between generations of selection.

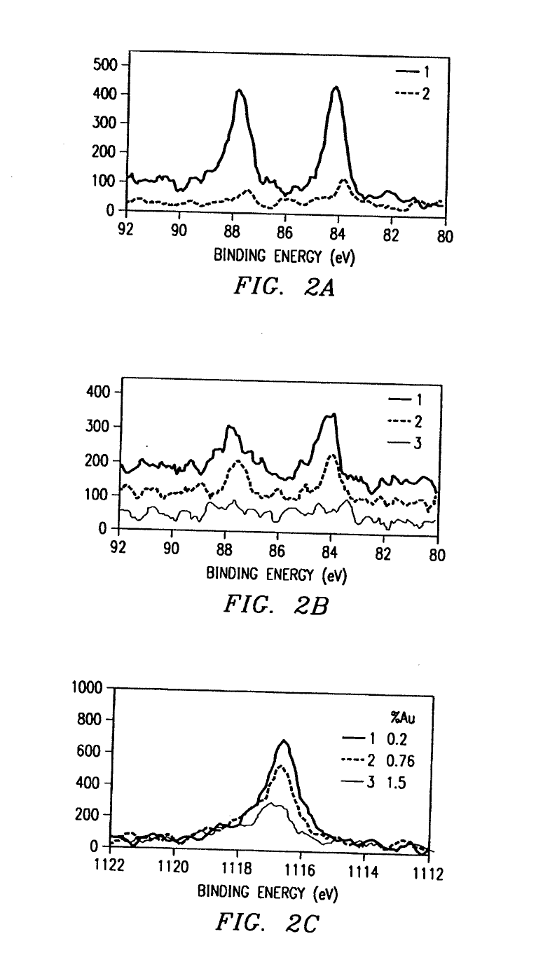

Phage, tagged with streptavidin-labeled 20-nm colloidal gold particles bound to the phage through a biotinylated antibody to the M13 coat protein, were used for quantitative assessment of specific binding. X-ray photoelectron spectroscopy (XPS) elemental composition determination was performed, monitoring the phage substrate interaction through the intensity of the gold 4f-electron signal (FIGS. 2a-c). Without the presence of the G1-3 phage, XPS confirmed that the antibody and the gold streptavidin did not bind to the GaAs(100)substrate. The gold-streptavidin binding was, therefore, specific to the peptide expressed on the phage and an indicator of the phage binding to the substrate. Using XPS it was also found that the G1-3 sequence isolated from GaAs(100) bound specifically to GaAs(100) but not to Si(100)(see FIG. 2a). In a complementary fashion the S1 clone, screened against the (100) Si surface, showed poor binding to the (100) GaAs surface.

Some GaAs sequences also bound the surface of InP (100), another zinc-blende structure. The basis of the selective binding, whether it is chemical, structural or electronic, is still under investigation. In addition, the presence of native oxide on the substrate surface may alter the selectivity of peptide binding.

The preferential binding of the G1-3 clone to GaAs(100), over the (111)A (gallium terminated) or (111)B (arsenic terminated) face of GaAs was demonstrated (FIG. 2b, c). The G1-3 clone surface concentration was greater on the (100) surface, which was used for its selection, than on the gallium-rich (111)A or arsenic-rich (111)B surfaces. These different surfaces are known to exhibit different chemical reactivities, and it is not surprising that there is selectivity demonstrated in the phage binding to the various crystal faces. Although the bulk termination of both 111 surfaces give the same geometric structure, the differences between having Ga or As atoms outermost in the surface bilayer become more apparent when comparing surface reconstructions. The composition of the oxides of the various GaAs surfaces is also expected to be different, and this in turn may affect the nature of the peptide binding.

The intensity of Ga 2p electrons against the binding energy from substrates that were exposed to the G1-3 phage clone is plotted in 2c. As expected from the results in FIG. 2b, the Ga 2p intensities observed on the GaAs (100), (111)A and (111)B surfaces are inversely proportional to the gold concentrations. The decrease in Ga 2p intensity on surfaces with higher gold-streptavidin concentrations was due to the increase in surface coverage by the phage. XPS is a surface technique with a sampling depth of approximately 30 angstroms; therefore, as the thickness of the organic layer increases, the signal from the inorganic substrate decreases. This observation was used to confirm that the intensity of gold-streptavidin was indeed due to the presence of phage containing a crystal specific bonding sequence on the surface of GaAs. Binding studies were performed that correlate with the XPS data, where equal numbers of specific phage clones were exposed to various semiconductor substrates with equal surface areas. Wild-type clones (no random peptide insert) did not bind to GaAs (no plaques were detected). For the G1-3 clone, the eluted phage population was 12 times greater from GaAs(100) than from the GaAs(111)A surface.

The G1-3, G12-3 and G7-4 clones bound to GaAs(100) and InP(100) were imaged using atomic force microscopy (AFM). The InP crystal has a zinc-blende structure, isostructural with GaAs, although the In-P bond has greater ionic character than the GaAs bond. The 10-nm width and 900-nm length of the observed phage in AFM matches the dimensions of the M13 phage observed by transmission electron microscopy (TEM), and the gold spheres bound to M13 antibodies were observed bound to the phage (data not shown). The InP surface has a high concentration of phage. These data suggest that there are many factors involved in substrate recognition, including atom size, charge, polarity and crystal structure.

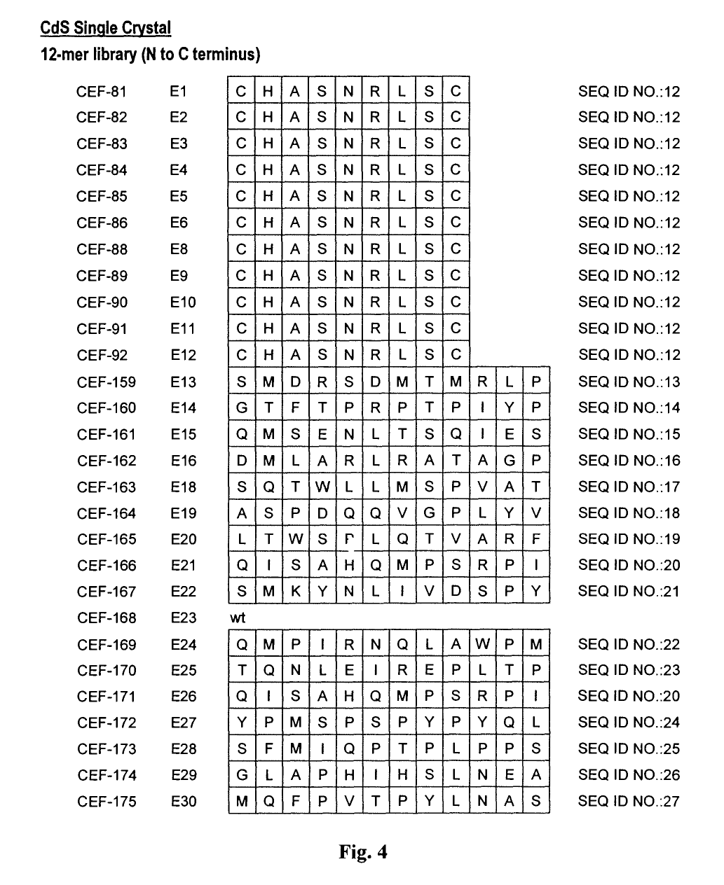

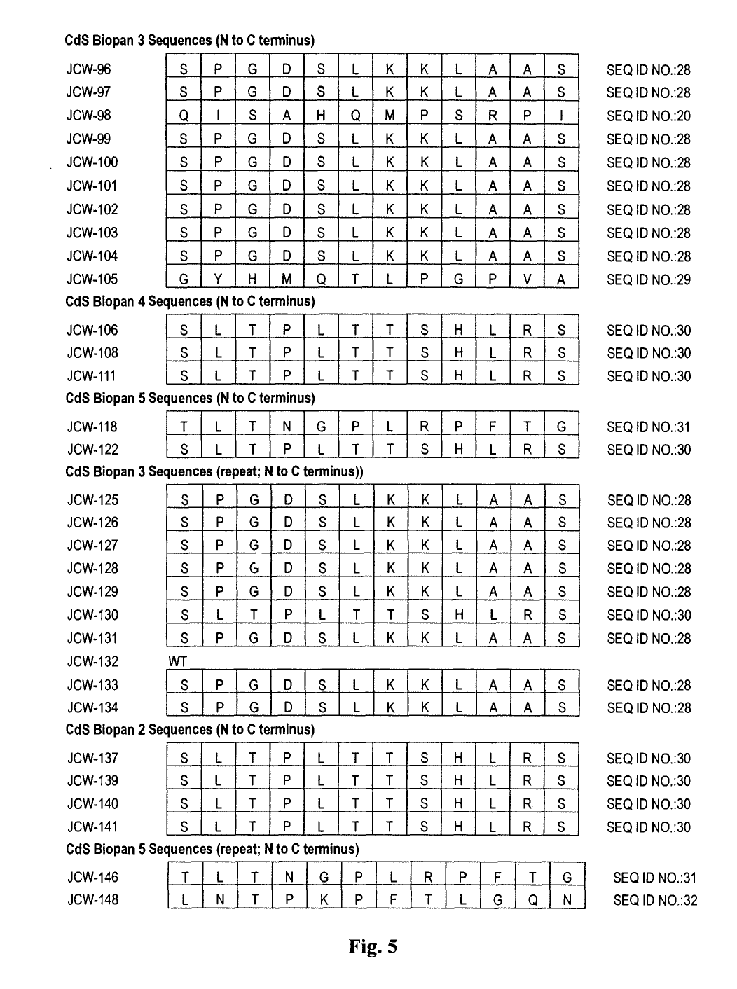

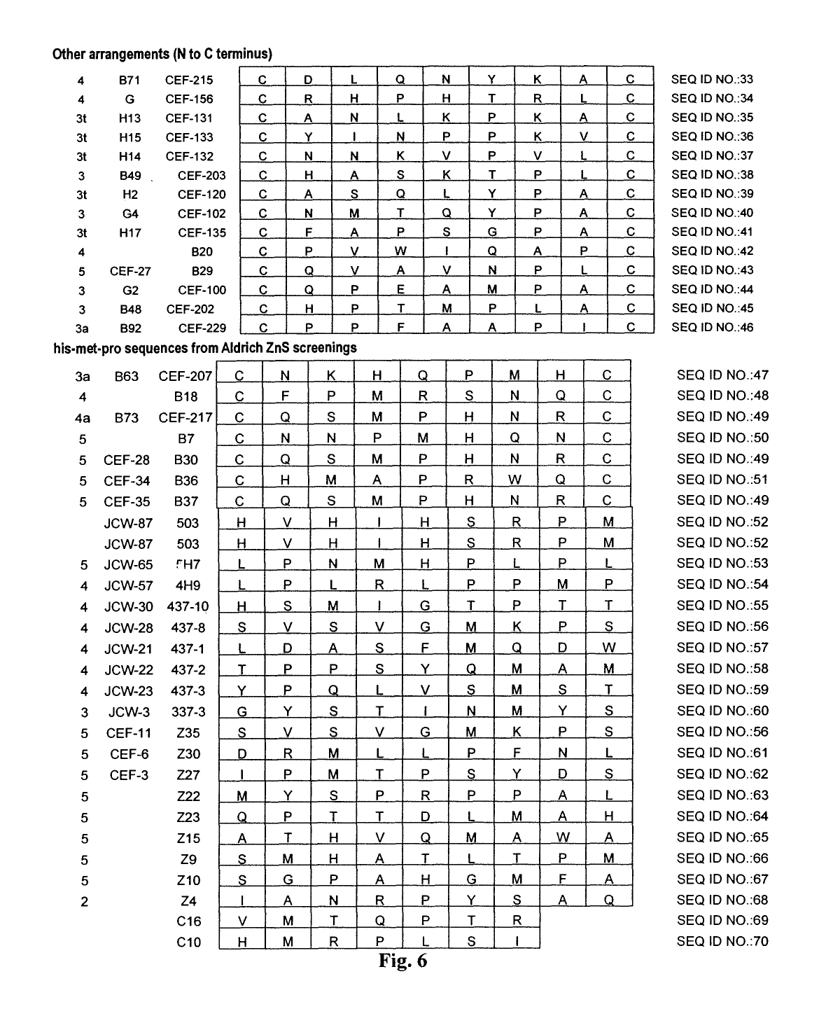

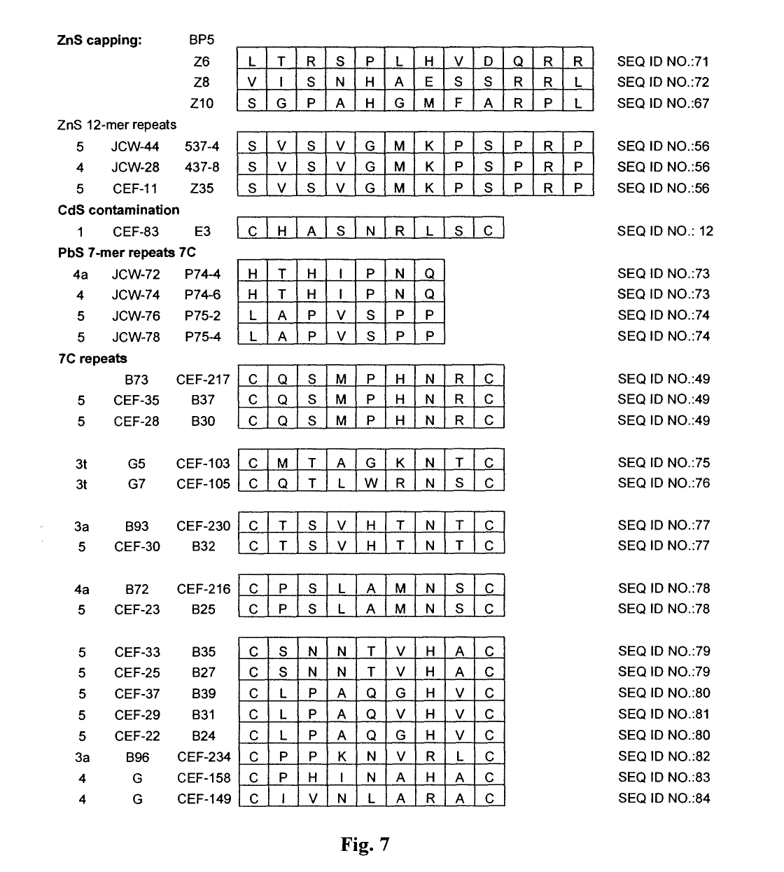

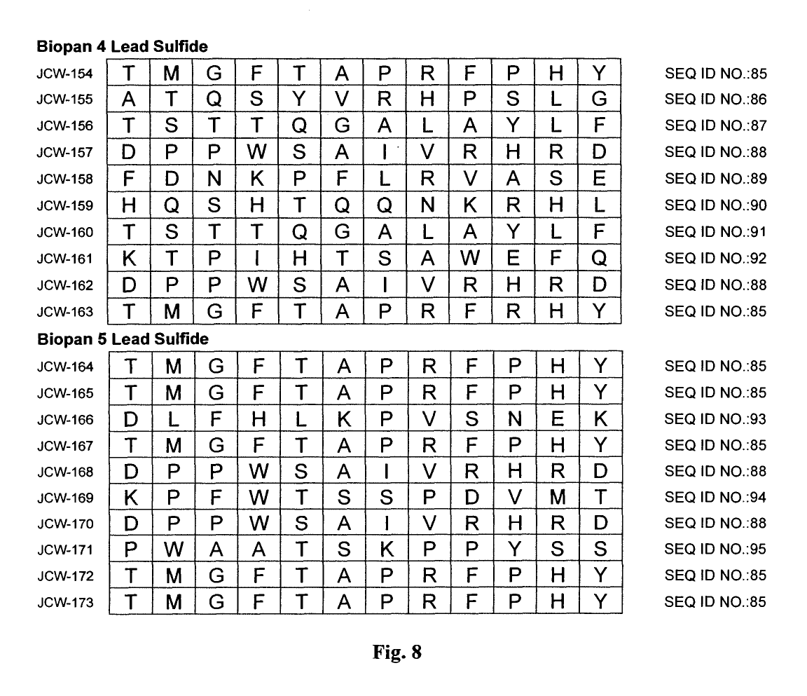

The G1-3 clone (negatively stained) is seen bound to a GaAs crystalline wafer in the TEM image (not shown). The data confirms that binding was directed by the modified pIII protein of G1-3, not through non-specific interactions with the major coat protein. Therefore, peptides of the present invention may be used to direct specific peptide-semiconductor interactions in assembling nanostructures and heterostructures (FIG. 4e).

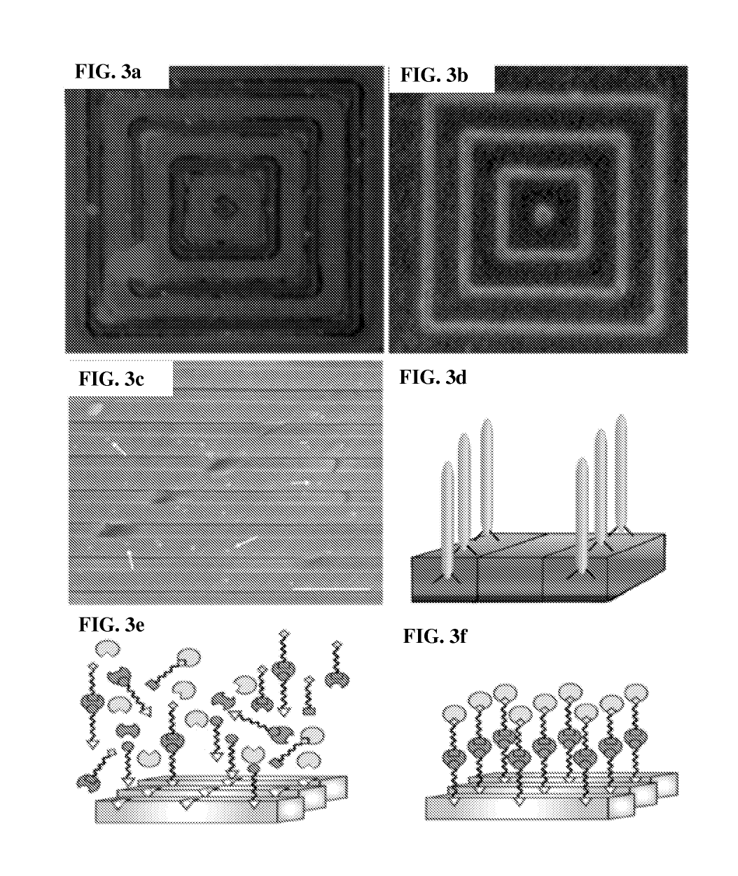

X-ray fluorescence microscopy was used to demonstrate the preferential attachment of phage to a zinc-blende surface in close proximity to a surface of differing chemical and structural composition. A nested square pattern was etched into a GaAs wafer; this pattern contained 1-μm lines of GaAs, and 4-μm SiO₂ spacings in between each line (FIGS. 3a, 3b). The G12-3 clones were interacted with the GaAs/SiO2 patterned substrate, washed to reduce non-specific binding, and tagged with an immuno-fluorescent probe, tetramethyl rhodamine (TMR). The tagged phage were found as the three red lines and the center dot, in FIG. 3b, corresponding to G12-3 binding only to GaAs. The SiO₂ regions of the pattern remain unbound by phage and are dark in color. This result was not observed on a control that was not exposed to phage, but was exposed to the primary antibody and TMR (FIG. 3a). The same result was obtained using non-phage bound G12-3 peptide.

The GaAs clone G12-3 was observed to be substrate-specific for GaAs over AlGaAs (FIG. 3c). AlAs and GaAs have essentially identical lattice constraints at room temperature, 5.66 A° and 5.65 A°, respectively, and thus ternary alloys of AlxGa1-xAs can be epitaxially grown on GaAs substrates. GaAs and AlGaAs have zinc-blende crystal structures, but the G12-3 clone exhibited selectivity in binding only to GaAs. A multilayer substrate was used, consisting of alternating layers of GaAs and of Al_{0.98}Ga_{0.02}As. The substrate material was cleaved and subsequently reacted with the G12-3 clone.

The G12-3 clones were labeled with 20-nm gold-streptavidin nanoparticles. Examination by scanning electron microscopy (SEM) shows the alternating layers of GaAs and Al_{0.98}Ga_{0.02}As within the heterostructure (FIG. 3c). X-ray elemental analysis of gallium and aluminum was used to map the gold-streptavidin particles exclusively to the GaAs layers of the heterostructure, demonstrating the high degree of binding specificity for chemical composition. In FIG. 3d, a model is depicted for the discrimination of phage for semiconductor heterostructures, as seen in the fluorescence and SEM images (FIGS. 3a-c).

The present invention demonstrates the power use of phage-display libraries to identify, develop and amplify binding between organic peptide sequences and inorganic semiconductor substrates. This peptide recognition and specificity of inorganic crystals has been demonstrated above with GaAs, InP and Si, and has been extended to other substrates, including GaN, ZnS, CdS, Fe₃O₄, Fe₂O₃, CdSe, ZnSe and CaCO₃ using peptide libraries by the present inventors. Bivalent synthetic peptides with two-component recognition (FIG. 4e) are currently being designed; such peptides have the potential to direct nanoparticles to specific locations on a semiconductor structure. These organic and inorganic pairs and potentially multivalent templates should provide powerful building blocks for the fabrication of a new generation of complex, sophisticated electronic structures.

### EXAMPLE I

### Peptide Creation, Isolation, Selection and Characterization

Peptide selection. The phage display or peptide library was contacted with the semiconductor, or other, crystals in Tris-buffered saline (TBS) containing 0.1% TWEEN-20, to reduce phage-phage interactions on the surface. After rocking for 1 h at room temperature, the surfaces were washed with 10 exposures to Tris-buffered saline, pH 7.5, and increasing TWEEN-20 concentrations from 0.1% to 0.5%(v/v) as selection rounds progressed. The phage were eluted from the surface by the addition of glycine-HCl (pH 2.2) for 10 minutes to disrupt binding. The eluted phage solution was then transferred to a fresh tube and then neutralized with Tris-HCl (pH 9.1). The eluted phage were tittered and binding efficiency was compared.

The phage eluted after third-round substrate exposure were mixed with their Escherichia coli ER2537 or ER2738 host and plated on LB XGal/IPTG plates. Since the library phage were derived from the vector M13mp19, which carries the lacZα gene, phage plaques were blue in color when plated on media containing Xgal (5-bromo-4-chloro-3-indoyl-β-D-galactoside) and IPTG (isopropyl-β-D-thiogalactoside). Blue/white screening was used to select phage plaques with the random peptide insert. Plaques were picked and DNA sequenced from these plates.

Substrate preparation. Substrate orientations were confirmed by X-ray diffraction, and native oxides were removed by appropriate chemical specific etching. The following etches were tested on GaAs and InP surfaces: NH₄OH:H₂O 1:10, HCl:H₂O 1:10, H₃PO₄:H₂O₂:H₂O 3:1:50 at 1 minute and 10 minute etch times. The best element ratio and least oxide formation (using XPS)for GaAs and InP etched surfaces was achieved using HCl:H₂O for 1 minute followed by a deionized water rinse for 1 minute. However, since an ammonium hydroxide etch was used for GaAs in the initial screening of the library, this etch was used for all other GaAs substrate examples. Si(100) wafers were etched in a solution of HF:H₂O 1:40 for one minute, followed by a deionized water rinse. All surfaces were taken directly from the rinse solution and immediately introduced to the phage library. Surfaces of control substrates, not exposed to phage, were characterized and mapped for effectiveness of the etching process and morphology of surfaces by AFM and XPS.

Multilayer substrates of GaAs and of Al_{0.98}Ga_{0.02} As were grown by molecular beam epitaxy onto (100) GaAs. The epitaxially grown layers were Si-doped (n-type) at a level of 5×10¹⁷ cm.

Antibody and Gold Labeling. For the XPS, SEM and AFM examples, substrates were exposed to phage for 1 h in Tris-buffered saline then introduced to an anti-fd bacteriophage-biotin conjugate, an antibody to the pIII protein of fd phage, (1:500 in phosphate buffer, Sigma) for 30 minute and then rinsed in phosphate buffer. A streptavidin/20-nm colloidal gold label (1:200 in phosphate buffered saline (PBS), Sigma) was attached to the biotin-conjugated phage through a biotin-streptavidin interaction; the surfaces were exposed to the label for 30 minutes and then rinsed several times with PBS.

X-ray Photoelectron Spectroscopy (XPS). The following controls were done for the XPS examples to ensure that the gold signal seen in XPS was from gold bound to the phage and not non-specific antibody interaction with the GaAs surface. The prepared (100) GaAs surface was exposed to (1) antibody and the streptavidin-gold label, but without phage, (2) G1-3 phage and streptavidin-gold label, but without the antibody, and (3) streptavidin-gold label, without either G1-3 phage or antibody.

The XPS instrument used was a Physical Electronics Phi ESCA 5700 with an aluminum anode producing monochromatic 1,487-eV X-rays. All samples were introduced to the chamber immediately after gold-tagging the phage (as described above) to limit oxidation of the GaAs surfaces, and then pumped overnight at high vacuum to reduce sample outgassing in the XPS chamber.

Atomic Force Microscopy (AFM). The AFM used was a Digital Instruments Bioscope mounted on a Zeiss Axiovert 100s-2tv, operating in tip scanning mode with a G scanner. The images were taken in air using tapping mode. The AFM probes were etched silicon with 125-mm cantilevers and spring constants of 20±100 Nm −1 driven near their resonant frequency of 200±400 kHz. Scan rates were of the order of 1±5 mms −1. Images were leveled using a first-order plane to remove sample tilt.

Transmission Electron Microscopy (TEM). TEM images were taken using a Philips EM208 at 60 kV. The G1-3 phage (diluted 1:100 in TBS) were incubated with GaAs pieces (500 mm) for 30 minute, centrifuged to separate particles from unbound phage, rinsed with TBS, and resuspended in TBS. Samples were stained with 2% uranyl acetate.

Scanning Electron Microscopy (SEM). The G12-3 phage (diluted 1:100 in TBS) were incubated with a freshly cleaved hetero-structure surface for 30 minute and rinsed with TBS. The G12-3 phage were tagged with 20-nm colloidal gold. SEM and elemental mapping images were collected using the Norian detection system mounted on a Hitachi 4700 field emission scanning electron microscope at 5 kV.

### EXAMPLE II

### Selection of Particle and Orientation Specific Peptides

It has been found that semiconductor nanocrystals exhibit size and shape-dependent optical and electrical properties may result in their potential applications in a variety of devices such as light emitting diode (LED), single electron transistor, photovoltaics, optical and magnetic memory, diagnostic markers and sensors. Control of particle size shape and phase is also critical in protective coatings, and pigments (car paints, house paints). To exploit these optical and electrical properties, it is necessary to synthesize crystallized semiconductor nanocrystals with, among other things, tailored size and shape.

The present invention includes compositions and methods for the selection and use of peptides that can: (1) recognize and bind technologically important materials with face specificity; (2) nucleate size constrained crystalline semiconductor materials; (3) control the crystallographic phase of nucleated nanoparticles; and (4) control the aspect ratio of the nanocrystals and, e.g, their optical properties.

Examples of materials used in this example were the Group II-VI semiconductors, which include materials such as: zinc sulfide, cadmium sulfide, cadmium selenide (CdSe) and zinc selenide (ZnSe). Size and crystal control could also be used with cobalt, manganese, iron oxides, iron sulfide, and lead sulfide as well as other optical and magnetic materials. Using the present invention, the skilled artisan can create inorganic-biological material building blocks that serve as the basis for a radically new method of fabrication of complex electronic devices, optoelectronic devices such as light emitting displays, optical detectors and lasers, fast interconnects, wavelength-selective switches, nanometer-scale computer components, mammalian implants and environmental and in situ diagnostics.

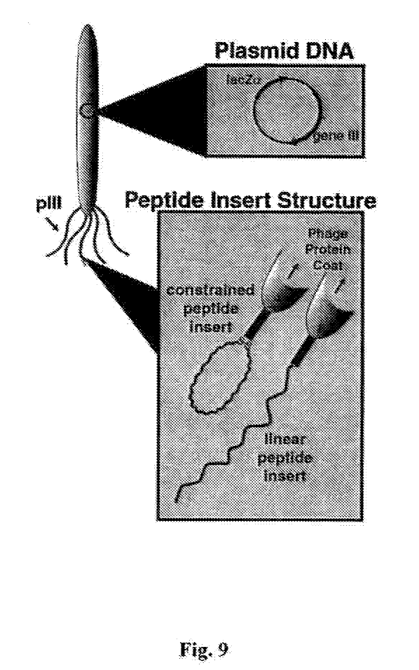

FIG. 9 depicts the expression of peptides using, e.g., a phage display library to express the peptides that will bind to the semiconductor material. Those of skill in the art of molecular biology will recognize that other expression systems may be used to “display” short or even long peptide sequences in a stable manner on the surface of a protein. Phage display may be used herein as an example. The phage-display library is a combinatorial library of random peptides containing between 7 and 12 amino acids. The peptides may be fused to, or form a chimera with, e.g., the pIII coat protein of M13 coliphage. The phage provided different peptides that were reacted with crystalline semiconductor structures. M13 pIII coat protein is useful because five copies of the pIII coat protein are located on one end of the phage particle, accounting for 10-16 nm of the particle. The phage-display approach provided a physical linkage between the peptide substrate interaction and the DNA that encodes that interaction. The semiconductor materials tested included ZnS, CdS, CdSe, and ZnSe.

To obtain peptides with specific binding properties, protein sequences that successfully bound to the specific crystal were eluted from the surface, amplified by, e.g., a million-fold, and reacted against the substrate under more stringent conditions. This procedure was repeated five times to select the phage in the library with the most specific binding. After, e.g., the third, fourth and fifth rounds of phage selection, crystal-specific phage were isolated and the DNA sequenced to decipher the peptide motif responsible for surface binding.

In one example of the present invention, two different peptides were found to nucleate two different phases of quantum dots. A linear 12-mer peptide, Z8, has been found that grows 3-4 nm particles of the cubic phase of zinc sulfide. A 7-mer disulfide constrained peptide, A7, has been isolated that grows nanoparticles of the hexagonal phase of ZnS. In addition, these peptides affect the aspect ratio (shape) of the nanoparticles grown. The A7 peptide has this “activity” while is it still attached to p3 of the phage or attached as a monolayer on gold. In addition phage/semiconductor nanoparticle nanowires wires were grown using an A7 fusion to the p8 protein on the virus coat. The nanoparticles grown on the phage coat show perfect crystallographic alignment of ZnS particles.

Peptides Controlling Nanoparticle Size, Morphology and Aspect Ratio. Phage that display a shape-controlling amino acid sequence were isolated, characterized and selected that specifically bind to ZnS, CdS, ZnSe and CdSe crystals. The binding affinity and discrimination of these peptides was tested and based on the results, peptides will be engineered for higher affinity binding. To conduct the tests, the phage library was screened against mm-size polycrystalline ZnS pieces. Binding clones were sequenced and amplified after third, fourth and fifth round selections. Sequences were analyzed and clones were tested for the ability of peptides that bind ZnS to nucleate nanoparticles of ZnS.

The clones designated Z8, A7 and Z10 clone were added to ZnS synthesis experiments to attempt to control ZnS particle size and monodispersity at room temperature in aqueous conditions. The ZnS-specific clones were interacted with Zn⁺² ions in millimolar concentrations of ZnCl₂ solution. The ZnS-specific peptide bound to the phage acts as a capping ligand, controlling crystalline particle size as ZnS is formed upon addition of Na₂S to the phage-ZnCl₂ solution.

Upon introduction of millimolar concentrations of Na₂S, crystalline material was observed to be in suspension. The suspensions were analyzed for particle size and crystal structures using transmission electron microscopy (TEM) and electron diffraction (ED). The TEM and ED data revealed that the addition of the ZnS-specific peptide bound to the phage clone affected the particle size of the forming ZnS crystals.

Crystals grown in the presence of the ZnS were observed to be approximately 5 nm in size and discrete particles. Crystals grown without the ZnS phage clones were much larger (>100 nm) and exhibited a range of sizes.

TABLE 1
Binding domains of ZnS specific clones.
Written Amino to Carboxy terminus

A7 NH2-Asn Asn Pro Met His Gln Asn Cys-COO-
(SEQ ID NO.: 105)
Z8-NH2-Val Ile Ser Asn His Ala Glu Ser Ser Arg
Arg Leu-COO-(SEQ ID NO.: 72)
Z10 NH2-Ser Gly Pro Ala His Gly Met Phe Ala Arg
Pro Leu-COO-(SEQ ID NO.: 106)

TABLE 2
Binding domains of CdS specific clones.
Written Amino to Carboxy terminus

El: Cys His Ala Ser Asn Arg Leu Ser Cys
(SEQ ID NO.: 12)
E14: Gly Thr Phe Thr Pro Arg Pro Thr Pro Ile Tyr
Pro (SEQ ID NO.: 14)
E15: Gln Met Ser Glu Asn Leu Thr Ser Gln Ile Glu
Ser (SEQ ID NO.: 15)
JCW-96: Ser Pro Gly Asp Ser Leu Lys Lys Leu Ala
Ala Ser (SEQ ID NO.: 28)
JCW-106: Ser Leu Thr Pro Leu Thr Thr Ser His Leu
Arg Ser (SEQ ID NO.: 30)
JCW-137: Ser Leu thr Pro Leu Thr Thr Ser His Leu
Arg Ser (SEQ ID NO.: 30)
JCW-182: Cys Thr Tyr Ser Arg Leu His Leu Cys
(SEQ ID NO.: 107)
JCW-201: Cys Arg Pro Tyr Asn Ile His Gln Cys
(SEQ ID NO.: 108)
JCW-205: Cys Pro Phe Lys thr Ala Phe Pro Cys
(SEQ ID NO.: 109)

The peptide insert structure expressed during phage generation, e.g., a 12 mer linear and 7 mer constrained libraries with a disulfide bond have been used, with similar results.

Peptides selected for ZnS using a 12 amino acid linear library verses a 7 amino acid constrained loop library had a significant effect on both the crystal structure of ZnS and the aspect ratio of the ZnS nanocrystals.

High resolution lattice images of nanoparticles grown in the presence of phage displaying 12 mer linear peptides that had been selected for ZnS revealed the crystals grew 3-4 nm spheres (1:1 aspect ratio) of the cubic (zinc-blende) form of ZnS. In contrast, the 7 mer constrained peptides selected to bind ZnS grew elliptical particles and wires (2:1 aspect ratio and 8:1 aspect ratio) of the hexagonal (wurzite) form of ZnS. Thus, the nanocrystal properties could be engineered by adjusting the length and sequence of the peptide. Further, electron diffraction patterns of the crystals revealed that peptides from different clones can stabilize the two different crystal structures of ZnS. The Z8 12 mer peptide stabilized the zinc-blende structure and the A7 7 mer constrained peptide stabilized the wurzite structure.

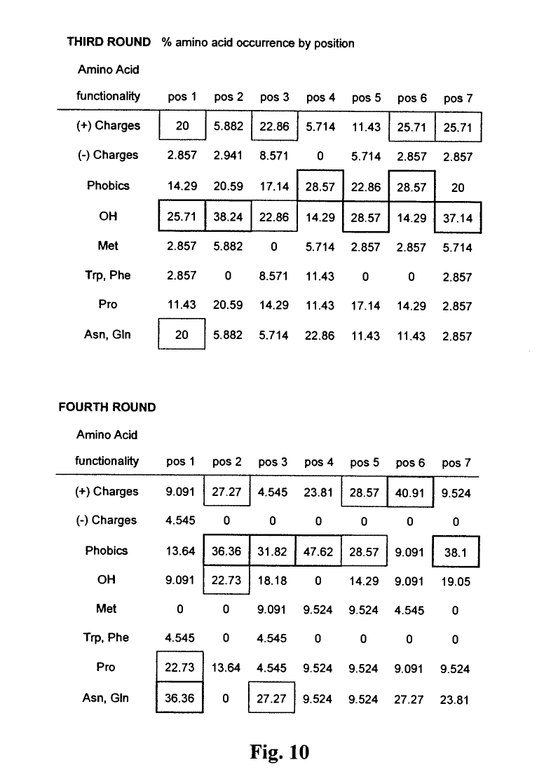

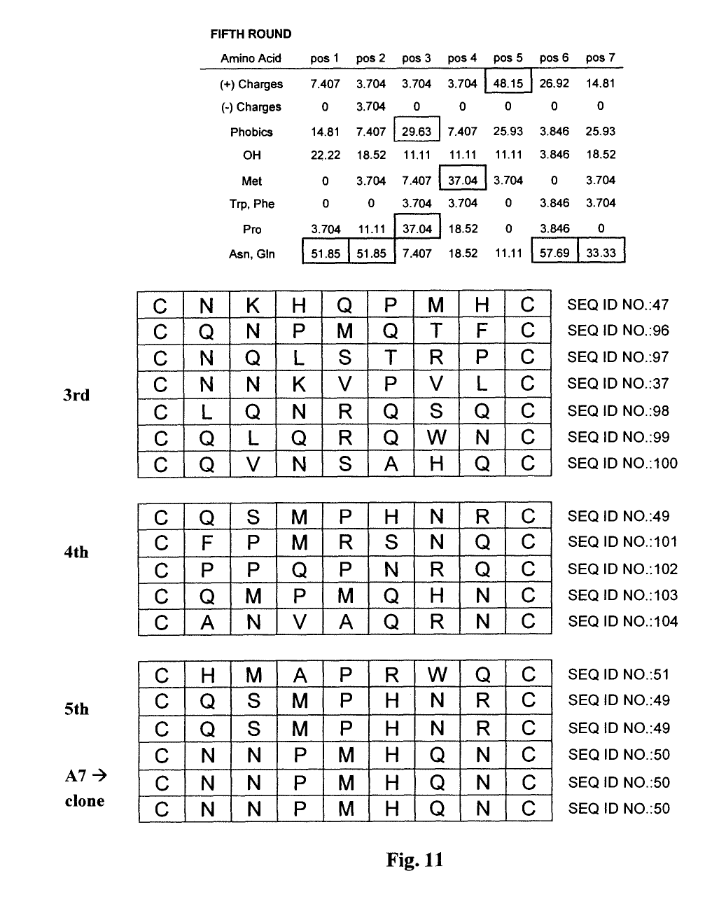

FIG. 10 shows the sequence evolution for ZnS peptides after the third, fourth and fifth rounds of selection. For peptide selection with the 7 mer constrained library, the best binding peptide sequence was obtained by the fifth round of selection. This sequence was named A7. Approximately thirty percent of the clones isolated after the fifth round of selection had the A7 sequence. The ASN/GLN at position number 1 was found to be significant starting from the third round of selection. In the fourth round of selection, the ASN/GLN also became important in position numbers 6 and 7. This importance increased in round 5. Throughout rounds 3, 4, and 5, a positive charge became prominent at position 5. FIG. 11 depicts the amino acid substitutions after the fifth round of selection in accordance with the present invention.

Site-directed mutagenesis is being conducted in the A7 sequence to test for a change in binding affinity. Mutations being tested include: position 3: his ala; position 4: met ala; position 2: gln ala; and position 6: asn ala. These changes may be made to the peptide concurrently, individually or in combinations.

The amino acid sequence motif defined for ZnS binding is, therefore: Written Amino to Carboxy terminus:

Amide-amide-Xaa-Xaa-positive-amide-amide;
or
(SEQ ID NO.: 110)
ASN/GLN-ASN/GLN-PRO-MET-HIS-ASN/GLN-ASN/GLY.

The clones isolated for ZnS through binding studies showed preferential interaction to ZnS, the substrate against which they had been raised, versus foreign clones and foreign substrates.

Interactions of different clones with different substrates such as FeS, Si, CdS and ZnS showed that the clones isolated through binding studies for ZnS showed preferential interaction to the ZnS against which they had been raised. Briefly, after washings and infection, phage titers were counted and compared. For Z8 and Z10, no titer count was evident on any substrate except ZnS. Wild-type clones with no peptide insert were used as a control to verify that the engineered insert had indeed mediated the interaction of interest. Without the peptide, no specific binding occurred, as was evidenced by a titer count of zero.

Using the same binding method that was used for, several different ZnS clones were compared to each other. Clones having different peptide inserts at the same concentration were interacted with a similar sized piece of ZnS for one hour. The substrate-phage complex was washed repeatedly, and the bound phage was eluted by changing the pH. The eluate was infected into bacteria and the plaques were counted after an overnight incubation. Z8 showed the greatest affinity for the ZnS of the 12 mer linear peptides selected. The wild-type did not show binding to the ZnS crystal. The Z8, Z10 and the wild-type peptides did not bind to the Si, FeS or CdS crystals.

The synthesis and assembly of nanocrystals on peptide functionalized surfaces was determined. The A7 peptide was tested alone for the ability to control the structure of ZnS. The A7 peptide, which specifically selected and grew ZnS crystals when attached to the phage, was applied in the form of a functionalized surface on a gold substrate that could direct the formation of ZnS nanocrytals from solution. A process that is used to prepare self-assembled monolayer was employed to prepare a functionalized surface.

To determine the ability and selectivity of A7 in the formation of ZnS nanocrystals, different kinds of surfaces with different surface chemistry on the gold substrate were interfaced with ZnS precursor solution. ZnCl₂ and Na₂S were used as the ZnS precursor solutions. CdS precursor solution of CdCl₂ and Na₂S was used as the CdS source. The crystals that formed on the four surfaces were characterized by SEM/EDS and TEM observation.

Control surface 1 consisted of a blank gold substrate. After being aged for 70 h in either ZnS solution or CdS solution, crystals formation was not observed. Control surface 2 consisted of a 2-mercaptoethyamine self-assembled monolayer on a gold substrate. This surface could not induce the formation of ZnS and CdS nanocrystals. In a few places, ZnS precipitates were observed. For the CdS system, sparsely distributed 2 micron CdS crystals were observed. Precipitation of these crystals occurred when the concentrations of both Cd⁺² and S⁻² were at 1×10⁻³ M.

The third surface tested was an A7-only functionalized gold surface. This surface was able to direct the formation of 5 nm ZnS nanocrystals, but could not direct the formation of CdS nanocrystals.

The fourth surface tested was an A7-amine functionalized gold surface that was prepared by aging control surface 2 in A7 peptide solution. The ZnS crystals formed on this surface were 5 nm and the CdS crystals were 1-3 μm. The CdS crystals could also be formed on the amine-only surface.

From the results of the four surfaces, the A7 peptide could direct the formation of ZnS nanocrystals for which it was selected, but could not direct the formation of CdS nanocrystals. Further, peptides selected against CdS could nucleate nanoparticles of CdS.

The peptides that could specifically nucleate semiconductor materials were expressed on the p8 major coat protein of M13. The p8 proteins are known to self-assemble into a highly oriented, crystalline protein coat. The hypothesis was that if the peptide insert could be expressed in high copy number, the crystalline structure of the p8 protein would be transferred to the peptide insert. It was also predicted that if the desired peptide insert maintained a crystal orientation relative to the p8 coat, then the crystals that nucleated from this peptide insert should grow nanocrystals that are crystallographically related. This prediction was tested and confirmed using high resolution TFM.

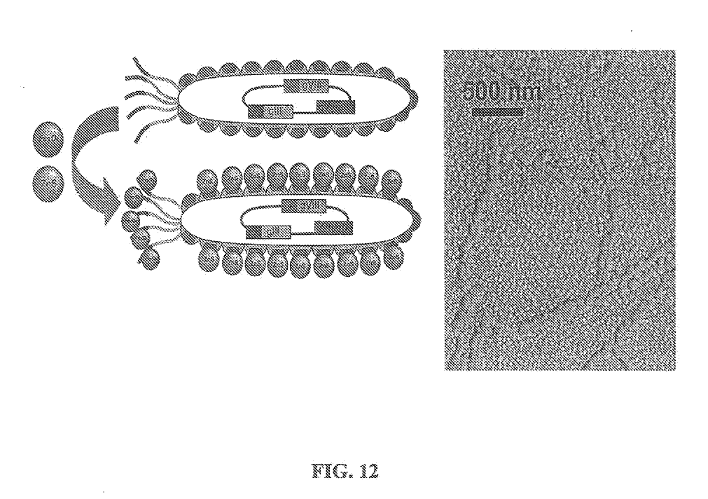

FIG. 12 shows a schematic diagram of the p8 and p3 inserts used to form nanowires. ZnS nanowires were be made by nucleating ZnS nanoparticles off of the A7 peptide fusion along the p8 protein coat of M13 phage. The ZnS nanoparticles coated the surface of the phage. The HR TEM image of ZnS nucleated on the coats of M13 phage that have the A7 peptide insert within the p8 protein showed that the nanocrystals nucleated on the coat of the phage were perfectly oriented. It is not clear whether the phage coat was a mixture of the p8-A7 fusion coat protein and the wild-type p8 protein. Similar experiments were performed with the Z8 peptide insert, and although the ZnS crystals were also nucleated along the phage, they were not orientated relative to each other.

Atomic force microscopy (AFM) was used to imagine the results, which indicated that the p8-A7 self-assembling crystals coated the surface of the phage, creating nanowires along the crest of the chimeric protein at the location of the A7 peptide sequence (data not shown). Nanowires were made by nucleating ZnS nanoparticles at the sites of the p8-A7 fusion along the coat of M13.

Nanocrystal nucleation of ZnS on the coat M13 phage that have the A7 peptide insert in the p8 protein was confirmed by high resolution TEM. Crystal nucleation was achieved despite the fact that some wild type p8 protein was found mixtured in with the p8-A7 fusion coat protein. The nanocrystals nucleated on the coat of the phage were perfectly orientated, as evidenced by lattice imaging (data not shown). The data demonstrates that peptides can be displayed in the major coat protein with perfect orientation conservation, and that these orientated peptides can nucleate orientated mondispersed ZnS semiconductor nanoparticles.

The cumulative data showed that some peptides could be displayed in the major coat protein with perfect orientation conservation and that these peptides could nucleate orientated ZnS semiconductor nanoparticles.

Peptide selection. The phage display or peptide library was contacted with the semiconductor, or other crystals, in Tris-buffered saline (TBS) containing 0.1% TWEEN-20, to reduce phage-phage interactions on the surface. After rocking for 1 hour at room temperature, the surfaces were washed with 10 exposures to Tris-buffered saline, pH 7.5, and increasing TWEEN-20 concentrations from 0.1% to 0.5%(v/v) as selection rounds progressed. The phage were eluted from the surface by the addition of glycine-HCl (pH 2.2) for 10 minutes to disrupt binding. The eluted phage solution was then transferred to a fresh tube and then neutralized with Tris-HCl (pH 9.1). The eluted phage were titred and binding efficiency was compared.

The phage eluted after the third-round of substrate exposure were mixed with an Escherichia coli ER2537 or ER2738 host and plated on Luria-Bertani (LB) XGal/IPTG plates. Since the library phage were derived from the vector M13mp19, which carries the lacZα gene, phage plaques, or infection events, were blue in color when plated on media containing Xgal (5-bromo-4-chloro-3-indoyl-β-D-galactoside) and IPTG (isopropyl-β-D-thiogalactoside). Blue/white screening was used to select phage plaques with the random peptide insert. DNA from these plaques was isolated and sequenced.

Atomic Force Microscopy (AFM). The AFM used was a Digital Instruments Bioscope mounted on a Zeiss Axiovert 100s-2tv, operating in tapping mode. The images were taken in air using tapping mode. The AFM probes were etched silicon with 125-mm cantilevers and spring constants of 20±100 Nm⁻¹ driven near their resonant frequency of 200±400 kHz. Scan rates were of the order of 1±5 mms⁻¹. Images were leveled using a first-order plane to remove sample tilt.

Transmission Electron Microscopy (TEM). TEM images were taken on JEOL 2010 and JEOL200CX transmission electron microscopes. The TEM grids used were carbon on gold. No stain was used. After the samples were grown, the reaction mixture was concentrated on molecular weight cut-off filters and washed four times with sterile water to wash away any excess ions or non-phage bond particles. After concentrating to 20-50 μl, the sample was then dried down on TEM or AFM specimen grids.

While this invention has been described in reference to illustrative embodiments, this description is not intended to be construed in a limiting sense. Various modifications and combinations of the illustrative embodiments, as well as other embodiments of the invention will be apparent to persons skilled in the art upon reference to the description. It is therefore intended that the appended claims encompass any such modifications or embodiments.

1.-154. (canceled)

155. A composition comprising one or more peptide binding domains, wherein the domain selectively binds to a target semiconductor crystalline face and the domain selectively nucleates a nanocrystal of the target semiconductor crystal composition.

156. The composition according to claim 155, wherein the domain is bound to the nanocrystal of the target semiconductor crystal composition.

157. The composition according to claim 155, wherein the domain is bound to the nanocrystal of the target semiconductor composition, and the nanocrystal is aligned with other nanocrystals of the target semiconductor composition to form a nanowire of the target semiconductor composition.

158. The composition according to claim 155, wherein the domain is selected from a combinatorial library.

159. The composition according to claim 155, wherein the domain is between about 7 to 20 amino acids.

160. The composition according to claim 155, wherein the domain is fused to another molecule.

161. The composition according to claim 155, wherein the domain is part of a virus.

162. The composition according to claim 155, wherein the domain is part of a bacteriophage.

163. The composition according to claim 155, wherein the domain is part of a p3 or p8 modified bacteriophage.

164. The composition according to claim 155, wherein the domain is part of a protein.

165. The composition according to claim 155, wherein the domain functionalizes a substrate surface.

166. The composition according to claim 155, wherein the domain is attached on a substrate surface as a monolayer.

167. The composition according to claim 155, wherein the target semiconductor crystal is a single crystal.

168. The composition according to claim 155, wherein the target semiconductor crystal is part of a heterostructure crystal surface.

169. The composition according to claim 155, wherein the target semiconductor crystal is a III-V semiconductor crystal or a II-VI semiconductor crystal.

170. The composition according to claim 155, wherein the target semiconductor crystal is an inorganic semiconductor crystal.

171. The composition according to claim 155, wherein the target semiconductor crystal is a nanoparticle.

172. The composition according to claim 155, wherein the target semiconductor crystal has an oxide surface.

173. The composition according to claim 155, wherein the target semiconductor crystal comprises gallium arsenide, indium phosphide, gallium nitride, zinc sulfide, cadmium sulfide, aluminum arsenide, gallium stibinide, aluminum gallium arsenide, aluminum stibinide, cadmium selenide, zinc selenide, cadmium telluride, zinc selenide, indium arsenide, aluminum arsenide, and silicon.

174. The composition according to claim 155, wherein the nanocrystal exhibits size and shape dependent optical properties.

175. The composition according to claim 155, wherein the nanocrystal exhibits size and shape dependent electrical properties.

176. The composition according to claim 155, wherein the domain can nucleate the nanocrystal which shows controlled orientation.

177. A composition comprising:
one or more engineered peptide selective binding domains, wherein the domain selectively binds to a target crystalline face, and
one or more nanoparticles bound to the binding domain.

178. A composition comprising:
one or more viruses comprising one or more engineered peptide selective binding domains, wherein the domain selectively binds to a target crystalline face, and
one or more nanoparticles bound to the binding domain.

179. A composition comprising one or more engineered peptide selective binding domains, wherein the domain selectively binds to a target crystalline face, and which selectively nucleates a nanocrystal of the target crystalline composition.

180. A virus comprising (i) synthetic peptide domains fused to coat proteins of the virus, and (ii) nanocrystals bound to the peptide domains.

181. The virus according to claim 180, wherein the domains are fused to p3 proteins.

182. The virus according to claim 180, wherein the domains are fused to p8 proteins.

183. The virus according to claim 180, wherein the nanocrystals are inorganic nanocrystals.

184. The virus according to claim 180, wherein the nanocrystals are inorganic semiconductor nanocrystals.

185. A composition comprising a nanowire comprising oriented nanocrystals.

186. The composition according to claim 185, wherein the nanowire comprises oriented inorganic nanocrystals.

187. The composition according to claim 185, wherein the composition further comprises a virus, and the oriented nanocrystals are on the surface of the virus.

188. A method of forming a nanocrystal comprising the step of contacting the composition according to claim 155 with nanocrystal precursors to form nanocrystal.

189. A method of forming a nanowire comprising the step of contacting the composition according to claim 155 with nanocrystal precursors to form nanocrystals bound to the composition and oriented in the form of a nanowire.

190. A method of forming a nanowire comprising the step of contacting the composition according to claim 155 with nanocrystals to form nanocrystals bound to the composition and oriented in the form of a nanowire.

191. A method of patterning comprising the step of providing a patterned crystalline surface, and binding the compositions of claim 155 on the patterned crystalline surface.

192. A patterned substrate comprising a pattern on a surface, the pattern comprising the compositions according to claim 155.

193. A semiconductor device comprising a composition comprising one or more synthetic peptide selective binding domains, wherein the domain is selective for a target crystalline face of a target crystal, and the domain is bound to a crystal.
